(19) United States
(12) Patent Application Publication
(10) Pub. No.: US 2023/0114135 A1
(43) Pub. Date: Apr. 13, 2023

(54) SYSTEMS AND METHODS FOR MONITORING CAREGIVER BURNOUT RISK

(71) Applicant: Hill-Rom Services, Inc., Batesville, IN (US)

(72) Inventors: Neal E. Wiggermann, Batesville, IN (US); Craig M. Meyerson, Syracuse, NY (US); Jie Zhou, Batesville, IN (US); Lori A. Zapfe, Milroy, IN (US)

(21) Appl. No.: 17/962,145

(22) Filed: Oct. 7, 2022

Related U.S. Application Data
(60) Provisional application No. 63/253,245, filed on Oct. 7, 2021.

(51) Int. Cl. G06Q 10/06

(52) U.S. Cl. CPC G06Q 10/06398; G06Q 10/063116

(57) ABSTRACT

Various techniques for managing caregiver burnout risk. One exemplary method includes receiving, over one or more electronic communication networks from a plurality of clinical data sources in a clinical environment, data associated with a caregiver having a plurality of patients. The method includes calculating, by one or more processors based on the data associated with the caregiver, at least one of a workload burnout risk score, an emotional burnout risk score, and physical burnout risk score for the caregiver. The method includes calculating, by the one or more processors based on at least one of the workload burnout risk score, the emotional burnout risk score, and the physical burnout risk score for the caregiver, a burnout risk score for the caregiver. The method includes determining, by the one or more processers, that the burnout risk score is greater than a threshold, and generating a report based on a determination that the burnout risk score is greater than the threshold.

### PRIORITY APPLICATION

This non-provisional application claims priority to U.S. Provisional Application No. 63/253,245 filed Oct. 7, 2021 and entitled “SYSTEMS AND METHODS FOR MONITORING CAREGIVER BURNOUT RISK.” The entire content of the provisional application is incorporated herein by reference.

### TECHNICAL FIELD

This application relates to a computing system for monitoring and mitigating risk of caregiver burnout in a clinical environment.

### BACKGROUND

Due to the physical and emotional demands inherent in the healthcare field, burnout among caregivers, such as nurses, has long been a problem. Recent surveys have shown that a significant percentage of caregivers intend to leave the profession or change positions in the near term. Caregiver turnover costs are significant, and a lack of reliable caregiver staff increasingly consumes the attention of administrators and affects continuity of care for patients.

Burnout is a depletion of emotional energy and a feeling that one's emotional resources are inadequate to deal with work demands. The causes of burnout are multifactorial and include physical and emotional demands. Although many of these demands are inherent in the work pressures associated with caregiving, clinical facilities such as hospitals can take measures to help alleviate and mitigate burnout of caregivers. For example, good leadership practices and supervisory support have been found to protect against burnout. Professional education and training have also been shown to reduce the likelihood of burnout. Additionally, interventions such as job optimization, shift scheduling, and counseling, and wellness programs can help.

Although such practices can help to avoid caregiver burnout, they do not assist administrators (e.g., supervisors) in monitoring their caregiver staff and detecting signs of burnout in advance.

This disclosure is directed to addressing one or more such problems in the field.

### SUMMARY

One aspect of the disclosure is directed to a method for managing caregiver burnout risk. The method may include receiving, over one or more electronic communication networks from a plurality of clinical data sources in a clinical environment, data associated with a caregiver having a plurality of patients. The method may further include calculating, by one or more processors based on the data associated with the caregiver, at least one of a workload burnout risk score, an emotional burnout risk score, and physical burnout risk score for the caregiver. The method may further include calculating, by the one or more processors based on at least one of the workload burnout risk score, the emotional burnout risk score, and the physical burnout risk score for the caregiver, a burnout risk score for the caregiver. The method may further include determining, by the one or more processers, that the burnout risk score is greater than a threshold, and generating a report based on a determination that the burnout risk score is greater than the threshold.

Another aspect of the disclosure is directed to a computer-readable storage medium storing instructions that, when executed by one or more processors, cause the one or more processors to perform a method for managing caregiver burnout risk. The method may include receiving, over one or more electronic communication networks from a plurality of clinical data sources in a clinical environment, data associated with a caregiver having a plurality of patients. The method may further include calculating, based on the data associated with the caregiver, at least one of a workload burnout risk score, an emotional burnout risk score, and physical burnout risk score for the caregiver. The method may further include calculating, based on at least one of the workload burnout risk score, the emotional burnout risk score, and the physical burnout risk score for the caregiver, a burnout risk score for the caregiver. The method may further include determining that the burnout risk score is greater than a threshold, and generating a report based on a determination that the burnout risk score is greater than the threshold.

Yet another aspect is directed to a system for managing caregiver burnout risk. The system may include a transceiver configured to receive, over one or more electronic communication networks from a plurality of clinical data sources in a clinical environment, data associated with a caregiver having a plurality of patients. The system may further include one or more processors configured to calculate, based on the data associated with the caregiver, at least one of a workload burnout risk score, an emotional burnout risk score, and physical burnout risk score for the caregiver. The one or more processors may be further configured to calculate, based on at least one of the workload burnout risk score, the emotional burnout risk score, and the physical burnout risk score for the caregiver, a burnout risk score for the caregiver. The one or more processors may be further configured to determine that the burnout risk score is greater than a threshold, and to generate a report based on a determination that the burnout risk score is greater than the threshold.

### DESCRIPTION OF THE FIGURES

The following figures, which form a part of this disclosure, are illustrative of described technology and are not meant to limit the scope of the claims in any manner.

FIG. 1 illustrates an example of a clinical environment, consistent with the disclosed embodiments;

FIG. 2 shows a representation of an exemplary burnout risk management system within the clinical environment of FIG. 1, consistent with the disclosed embodiments;

FIG. 3 shows an exemplary burnout dashboard graphical user interface (GUI) provided by the burnout risk management system, consistent with the disclosed embodiments;

FIG. 4 is a flowchart of an exemplary burnout risk management method performed by the burnout risk management system, consistent with the disclosed embodiments; and

FIG. 5 is a flowchart of an exemplary burnout management method performed by the burnout risk management system, consistent with the disclosed embodiments.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

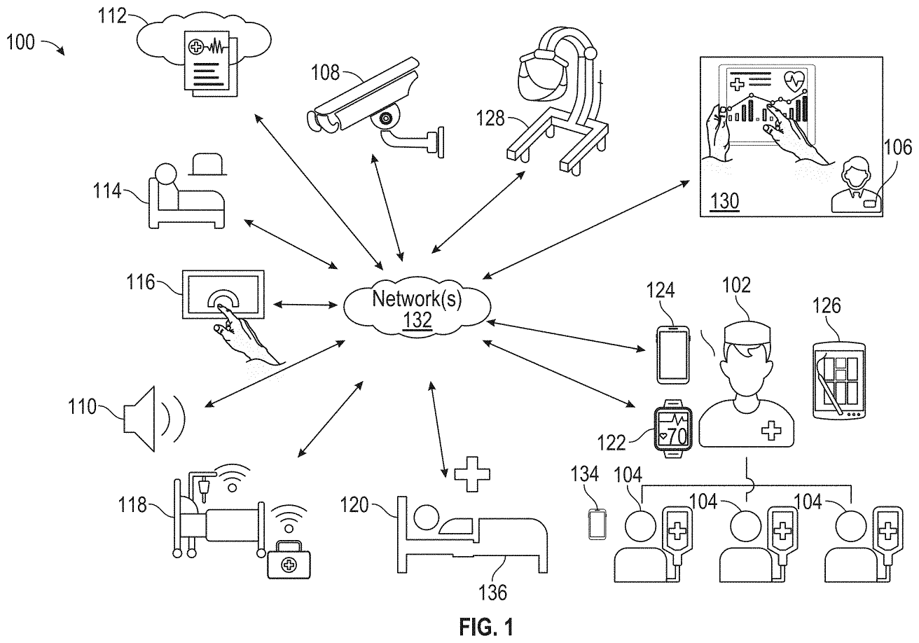

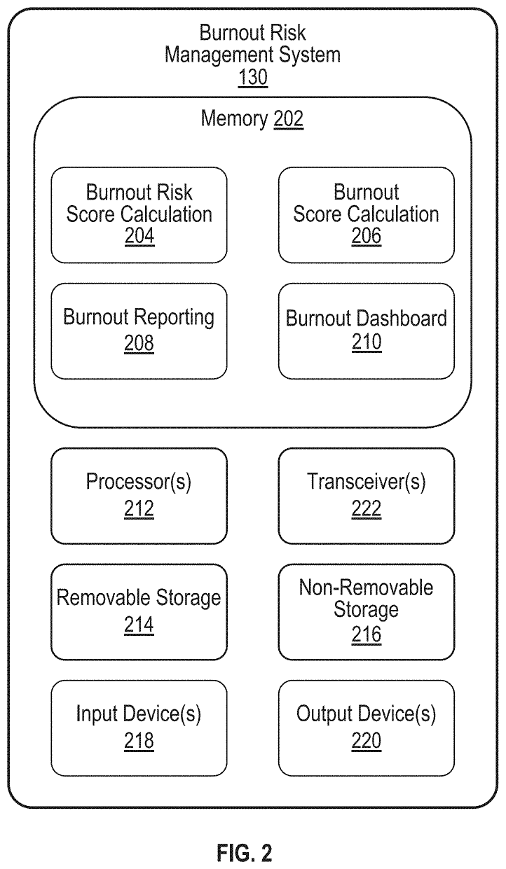

FIG. 1 illustrates an example clinical environment 100 for monitoring caregiver burnout risk in a clinical environment 100, such as a hospital, medical clinic, hospice, or any other type of managed care facility. As explained below, clinical environment 100 may include systems and methods that monitor the clinical environment 100 in which one or more caregiver(s) 102—such as a nurse, physician, physician's assistant, nurse practitioner, a physical therapist, or other clinical staff—works and cares for one or more patients 104. For example, as shown in FIG. 2 discussed in detail below, a burnout risk management system 130 may monitor clinical environment 100 and include a burnout risk score calculation engine 204 configured to determine a workload burnout risk score, an emotional burnout risk score, and/or a physical burnout risk score for caregiver(s) 102. The workload burnout risk score may indicate the likelihood that the caregiver(s) 102 will burn out based on recent workload conditions experienced on the job, such as the number of patients 104 under the caregiver(s) care and/or the medical complexity of those patients. The emotional burnout risk score, on the other hand, may indicate the likelihood that the caregiver(s) 102 will burn out based on recent emotional conditions experienced on the job, such as patient deaths or negative interactions with patient family members. And the physical burnout risk score may similarly indicate the likelihood that the caregiver(s) 102 will burn out based on recent physical conditions experienced on the job, such as walking long distances, standing for long periods of time, and/or lifting or otherwise manipulating immobile patients 104. Based on the workload burnout risk score, the emotional burnout risk score, and/or the physical burnout risk score, the burnout risk score calculation engine 204 determine a burnout risk score for caregiver(s) 102. The burnout risk score may serve as an overall indicator of the likelihood that the caregiver(s) 102 will burn out based on a combination of the constituent workload, emotional, and/or physical risk scores.

Burnout risk management system 130 may be further configured to, based on the burnout risk score, perform one or more actions to help an administrator 106 responsible for the clinical staff including caregiver(s) 102—such as a supervisor—mitigate and address the burnout risk. For example, as shown in FIG. 2 discussed below, burnout risk management system 130 may include a burnout reporting engine 208 configured to send a report to the administrator based on the calculated burnout risk score for a caregiver being greater than a threshold, allowing the administrator to take preventative action to mitigate and/or prevent burnout and/or the effects thereof. Additionally, as discussed below, burnout risk management system 130 may include a burnout dashboard engine 210, providing a graphical dashboard giving the administrator 106 an overall visual picture of the burnout risk of the caregiver(s) for which the administrator is responsible, which may further enable the administrator 106 to take preventative action.

As shown in FIG. 2, burnout risk management system 130 may further include a burnout score calculation engine 206 configured to calculate an actual burnout score for caregiver(s) 102. In contrast to the burnout risk score which indicates a likelihood of potential future burnout, the actual burnout score may indicate a degree to which a caregiver 102 is exhibiting signs or behavior of burnout—i.e., a degree to which the caregiver is currently burned out. Burnout reporting engine 208 may be further configured to send a report to administrator 106, for example, based on a determination that the actual burnout score is greater than a threshold.

Clinical environment 100 may include a plurality of clinical data sources used to monitor the clinical environment 100 and determine the risk of burnout of caregiver(s) 102, as discussed below. Such clinical data sources may include any devices or systems typically used in a clinical environment 100 in the ordinary course of business. As examples, the clinical data sources may include one or more camera(s) 108, microphone(s) 110, an electronic health record (EHR) system 112, a caregiver call system 114, a caregiver communication system 116, a personnel/asset tracking system 118, patient bed(s) 120, wearable device(s) 122, mobile device(s) 124, clinical device(s) 126, and/or patient lift system(s) 128 in communication with a burnout risk management system 130 over one or more communication network(s) 132.

Camera(s) 108 may be configured to capture images of areas within the clinical environment 100, such as rooms, curtained areas, hallways, operating rooms, or other such areas. The captured images may include one or more objects. As used herein, the term “object,” and its equivalents, may refer to a portion of an image that can be interpreted as a single unit and which depicts a single subject depicted in the image. Examples of subjects include machines, devices, equipment, as well as individuals (e.g., caregivers, patients, or visitors) depicted in the images.

For example, camera(s) 108 may monitor patients 104, caregiver(s) 102, visitors or other individuals in the clinical environment 100. In some implementations, a patient 104 may be assigned to, or otherwise associated with, the area monitored by camera(s) 108. For example, a patient 104 may at least temporarily reside in a room captured by camera(s) 108. In various cases, at least some of the images captured by the camera(s) 108 may depict the patient 104, caregiver(s) 102 when interacting with the patient 104, visitors of the patient 104 interacting with the patient 104 and/or caregiver(s) 102, etc. Camera(s) 108 may communicate the captured images, e.g., as digital or analog electronic video signals, over communication network(s) 132 to burnout risk management system 130.

Like camera(s) 108, microphone(s) 110 may be positioned in certain areas in the clinical environment 100—such as rooms, curtained areas, hallways, operating rooms, ICU rooms—and capture voices, noises, and/or other sounds made within those areas. For example, microphone(s) 110 may capture the voices of caregiver(s) 102 when interacting with each other or other clinical staff, with patients 104, with visitors of patients 104, with medical equipment technicians, and/or with other individuals present in the clinical environment 100. Microphone(s) 110 may communicate the capture audio, for example, as digital or analog electronic audio signals, over communication network(s) 132 to burnout risk management system 130.

EHR system 112 may be a computing system configured to receive, store, and transmit patient data in electronic health records for patients 104. As used herein, the terms “electronic medical record,” “EMR,” “electronic health record,” “EHR,” and their equivalents, may refer to a data indicating previous or current medical conditions, diagnostic tests, or treatments of a patient 104. The patient data in an EHR may relate to demographics of a patient 104, parameters of a patient 104 (e.g., height, weight, and age), vital signs of a patient 104, notes from one or more medical/health appointments attended by a patient 104, medications prescribed or administered to a patient 104, therapies (e.g., surgeries, outpatient procedures, etc.) administered to a patient 104, results of diagnostic tests performed on a patient 104, identification information (e.g., a name, birthdate, etc.) of a patient 104, identification information of caregiver(s) 102 treating patients 104, or any combination thereof.

EHR system 112 may be accessible over communication network(s) 132 by computing devices operated by users. For example, a caregiver 102 may use clinical device 126 or mobile device 124 running a clinical application to access EHRs of patients 104, input patient information into EHRs of patients 104, or retrieve patient data from EHRs of patients 104. Additionally, EHR system 112 may be configured to transmit EHRs and/or patient data to burnout risk management system 130 either automatically or in response to requests received from burnout risk management system 130 over network(s) 132.

Caregiver call system 114 may be any system or device accessible to, and operated by, patients 104 to page, alert, or otherwise call a caregiver 102 remotely when they need help. For example, caregiver call system 114 may include a wired or wireless control pad having one or more buttons enabling a patient 104 to remotely call caregiver 102 from bed, or from standing or walking about the patient's room, when the patient needs assistance. In some cases, caregiver call system 114 may include a microphone and speaker and/or a display to enable a patient 104 to audibly and/or visually communicate with caregiver 102 over network(s) 132 when calling a caregiver 102.

In some embodiments, when a patient 104 alerts the patient's caregiver(s) 102 of the need for help, caregiver call system 114 may generate a caregiver call record. The record may include data about the call, such as the time/date of the call, identification of the patient making the call, identification of patient's caregiver(s) 102 responsible for responding to the call at the time it was requested, and/or other such data. Additionally, in some implementations, caregiver call system 114 may communicate the caregiver call records to EHR system 112 and/or to burnout risk management system 130 over network(s) 132. In some embodiments, caregiver call system 114 may communicate a caregiver call record to EHR system 112 and/or to burnout risk management system 130 automatically when the call is made. In other embodiments, caregiver call system 114 may store the caregiver call records (e.g., in memory or a data storage device) and transmit the stored records to EHR system 112 and/or to burnout risk management system 130 periodically and/or upon receiving a request over communication network(s) 132.

Caregiver communication system 116 may be a computing system allowing patients 104 to communicate with their caregiver(s) 102, caregivers 102 to communicate with other caregivers 102 and/or other clinical staff, and/or caregivers 102 to communicate with family members of patient(s) 104 over communication network(s) 132. For example, caregiver communication system 116 may include a patient terminal 134, such as a mobile device carried by a patient 104 or a dedicated mounted on the patient's bed or in the patient's room. Using patient terminal 134, patient 104 may communicate with caregiver(s) 102 on their mobile device 124 and/or clinical device 126, and vice versa. Similarly, caregiver communication system 116 may allow caregivers 102 to communicate with other caregivers 102 or staff, with patients 104, and/or with family members of patients 104 on their respective mobile device(s) 124 and/or clinical devices 126.

In some embodiments, caregiver communication system 116 may allow caregivers 102 to send messages (e.g., instant messages or text messages) to other caregiver(s) 102 and/or staff, to patients 104, and/or to family members of patients 104. Similarly, caregiver communication system 116 may allow patients 104 to send messages to their caregiver(s) 102 and allow family members of patients 104 to send messages to caregiver(s) 102 responsible for those patients 104. When a message is sent, caregiver communication system 116 may generate a message record containing information about the message. For example, the message record may include the time/date of the message, an identification of individual sending the message (i.e., caregiver 102, patient 104, or family member), an identification of the individual receiving the message (i.e., caregiver 102, patient 104, or family member), the content of the message, or other such information. Caregiver communication system 116 may communicate the message records to EHR system 112 and/or to burnout risk management system 130 automatically when the messages are generated by caregiver(s) 102 and/or patients(2). In other embodiments, caregiver communication system 116 may store the message records and transmit the stored message records to EHR system 112 and/or to burnout risk management system 130 periodically and/or upon receiving a request over communication network(s) 132.

Similar to caregiver call system 114, caregiver communication system 116 may also be configured to allow patients 104 to remotely call their caregiver(s) 102 over network(s) 132. In some embodiments, caregiver communication system 116 may allow a patient 104 to identify a reason for the call (e.g., for help getting out of bed using the restroom). Caregiver call system 116 may maintain a status of any calls requested by patient 104. For example, a call may remain open or pending until responded to by caregiver(s) 102.

When a patient 104 calls the patient's caregiver(s) 102 using caregiver communication system 116, caregiver communication system 116 may generate a caregiver call record. The record may include data about the call, such as the time/date of the call, identification of the patient making the call, identification of patient's caregiver(s) 102 responsible for responding to the call at the time it was requested, the status of the call, the time it took for the caregiver to respond to the call, and/or other such data. Additionally, in some implementations, caregiver communication system 116 may communicate the caregiver call records to EHR system 112 and/or to burnout risk management system 130 over network(s) 132. In some embodiments, caregiver communication system 116 may communicate a caregiver call record to EHR system 112 and/or to burnout risk management system 130 automatically when the call is made. In other embodiments, caregiver communication system 116 may store the caregiver call records (e.g., in memory or a data storage device) and transmit the stored records to EHR system 112 and/or to burnout risk management system 130 periodically and/or upon receiving a request over communication network(s) 132.

In some implementations, caregiver communication system 116 may be configured to allow a patient 104 to remotely make a specific request of the patient's caregiver(s) 102. For example, providing input to patient terminal 134, a patient 104 may request a food item, a beverage, a pillow, a towel, an entertainment item (e.g., a book), a bath, a change of clothes, or other item or service. When a patient 104 makes a request of a caregiver 102, caregiver communication system 116 may make a caregiver request record including data about the request, such as the time/date of the request, identification of the patient making the request, identification of patient's caregiver(s) 102 responsible for responding to the request at the time it was requested, the status of the request, an identification of the particular item and/or service requested, and/or other such data. Additionally, caregiver communication system 116 may communicate the caregiver request records to EHR system 112 and/or to burnout risk management system 130 over network(s) 132. In some embodiments, caregiver communication system 116 may communicate a caregiver request record to EHR system 112 and/or to burnout risk management system 130 automatically when the call is made. In other embodiments, caregiver communication system 116 may store the caregiver request records locally and transmit the stored records to EHR system 112 and/or to burnout risk management system 130 periodically and/or upon receiving a request over communication network(s) 132.

Caregiver communication system 116 may maintain and track workload information for caregiver(s) 102. For example, the workload information may include a list of patients 104 for which a caregiver 102 is currently responsible. The workload information may further indicate an admission/discharge status of each of the caregiver's patients 104. For example, the admission/discharge status may include whether the caregiver 102 has been assigned a newly-admitted patient 104 on the caregiver's shift and/or whether the caregiver has a current patient 104 scheduled to be discharged on the caregiver's shift. The workload information may further include patient complexity information indicating whether each of the caregiver's patients 104 has any complicating factors such as a high risk/susceptibility to infection, a large size or weight, etc. that make caring for the patient more challenging. The workload information may further include staffing information about other clinical staff currently on shift with the subject caregiver 102, such as the number of clinical staff on the caregiver's shift and an indication whether this clinical staff are float/temporary staff.

Caregiver communication system 116 may communicate the caregiver workload information to burnout risk management system 130 over network(s) 132. In some embodiments, caregiver communication system 116 may communicate a caregiver request record to burnout risk management system 130 automatically in real-time. In other embodiments, caregiver communication system 116 may store the caregiver workload information locally and transmit the stored caregiver workload information to burnout risk management system 130 periodically, such as at the end of a day or shift, and/or upon receiving a request over communication network(s) 132 from burnout risk management system 130.

Personnel/asset tracking system 118 may be configured to track the location of caregiver(s) 102, other clinical staff, patients 104, and/or clinical equipment within the clinical environment. For example, tracking system 118 may be a component of a real-time location system (RTLS) configured to detect the location of various tags in the clinical environment 100. Tracking system 118 may include multiple fixed-position sensors configured to communicate with various tags disposed in the clinical environment 100. In some cases, the sensors receive a wireless signal (e.g., an NFC signal, radio frequency (RF) signal, etc.) broadcast from a tag. Tracking system 118 determines the times at which the wireless signal was received by the sensors from the tag. Based on a discrepancy of the times at which the signal was received, and the known positions of the sensors, the position of the tag can be derived (e.g., using triangulation). According to some implementations, the location of the tag is tracked over time as the tag broadcasts additional signals received by the sensors.

Tracking system 118 may be configured to determine the locations of a tag associated with caregiver(s) 102, with other clinical staff, with patients 104, with family members visiting patients 104, with clinical equipment (e.g., patient lift system 128, bed(s) 120, an MM machine, etc.), and/or with other objects. For instance, a tag associated with a patient 104 may be integrated into an identification bracelet worn by the patient 104, and the tag associated with a caregiver may be integrated into an ID badge, clothing, equipment, or other wearable item (e.g., wearable device 122) that is worn and/or carried by caregiver 102. Visitors of a patient 104 may be given a similar identification bracelet used to determine their location within the clinical environment 100. Similarly, tags may be associated with other individuals, equipment, and/or objects. For example, a tag may be associated with bed 120, with patient lift system 128, with a patient vital sign monitor, etc.

Accordingly, tracking system 118 may detect the location of such individuals, equipment, and/or other objects within the clinical environment 100 based on one or more respective integrated, attached, worn, or otherwise associated tags. Tracking system 118 may generate data indicating the location of at least one tag associated with at least one of caregiver(s) 102, other clinical staff, patients 104, family members visiting patients 104, clinical equipment, or with other objects in the clinical environment 100. Tracking system 118 may transmit the location data to burnout risk management system 130 over one or more communication network(s) 132. In this manner, burnout risk management system 130 may confirm the position, movement, and/or identity of an individual or object based on the location of a tag associated therewith, as indicated by tracking system 118.

In some embodiments, each patient 104 may have a bed 120 in the patient's respective room. A load sensor 136 associated with bed 120 may determine the presence or absence of the patient in bed 120 based on a measured load. When load sensor 136 detects that the patient has exited bed 120, a bed exit notification may be sent to burnout risk management system 130 over communication network(s) 132. In some embodiments, load sensor 136 may be connected to network(s) 132 and send the notification directly. In other cases, bed 120 may be a smart bed with an integrated load sensor 136, in which case bed 120 may be connected to communication network(s) 132 and send the notification to burnout risk management system 130.

Wearable device 122 may be worn by caregiver(s) 102 and detect or determine physiological and/or movement parameters of the caregiver. For example, as to physiological parameters, wearable device 122 may include a moisture sensor configured to detect the presence or absence of moisture (e.g., perspiration) on the body of the caregiver and/or a heart rate monitor configured to determine the caregiver's heart rate. And as to movement parameters, wearable device 122 may include an accelerometer/pedometer to determine the number of steps the caregiver has taken in a period of time, the distance walked by the caregiver in a period of time, the speed at which the caregiver moves (e.g., indicating walking or running), and/or the amount of time the caregiver has spent standing in place. As another example, a plurality of wearable devices 122 may be used to estimate the posture of a caregiver. Two or more inertial measurement units that include an accelerometer and gyroscope can be used to estimate the orientation of body segments. For example, the relative angle between two body segments such as the thigh and torso may help to determine whether the caregiver is sitting, standing, walking, or running.

Wearable device 122 may transmit information indicative of the detected or determined parameters to burnout risk management system 130 over communication network(s) 132. In some embodiments, wearable device 122 may transmit the parameter information continuously and/or in real time. In other examples, wearable device 122 may collect and store the parameter information locally, and may transmit the stored parameter information to burnout risk management system 130 periodically (e.g., hourly, daily, once per shift) or upon receiving a request. In yet other examples, wearable device 122 may transmit the parameter information upon detection of a certain event, such as the caregiver's heart rate, distance or number of steps walked in a certain period of time, amount of time standing or sitting in place, speed at which the caregiver is moving (e.g., running), the amount of time or number of times the caregiver moves at a high speed, and/or the amount or number of times the caregiver perspires exceeds a threshold.

Patient lift system 128 may be any device or system configured to lift and/or move patients 104 who are immobile due to injury, excessive bodyweight, doctor's orders, or other reasons. In some embodiments, patient lift system 128 and/or tracking system 118 may detect when a caregiver 102 uses patient lift system 128 to lift and/or move a patient 104. Each time patient lift system 128 is used to lift or more a patient 104, a record containing data about the use of the lift may be made. For example, the record may include the time/date of the use of the lift, an identification of the caregiver using the lift, etc. Additionally, patient lift system 128 and/or tracking system 118 may transmit the records to burnout risk management system 130 over communication network(s) 132 continuously, in real-time, periodically, or upon request.

Consistent with the disclosed embodiments, burnout risk management system 130 may be a computing system configured monitor the clinical environment 100 in which caregiver(s) 102 work and care for patients 104 based on the data received from the clinical data sources over communication network(s) 132. As discussed above, and in further detail below, burnout risk management system 130 may be configured to determine a workload burnout risk score, an emotional burnout risk score, and/or a physical burnout risk score for caregiver(s) 102 based on the received data. And, based on the constitute workload burnout risk score, the emotional burnout risk score, and/or the physical burnout risk score, the systems and methods may determine an overall burnout risk score for caregiver(s) 102. Burnout risk management system 130 may also include features and/or perform one or more actions to help administrator 106 responsible for the clinical staff, including caregiver(s) 102, mitigate and address the burnout risk. It will be appreciated that burnout risk management system 130 may be implemented in software, hardware (e.g., one or more computer processor), or a combination thereof. For instance, burnout risk management system 130 may be implemented and/or executed by one or more computing devices (e.g., servers, user terminals, etc.) located on the premises of clinical environment 100 and/or outside clinical environment 100.

Workload Burnout Risk Score

Based on the data received from the clinical data sources over communication network(s) 132, burnout risk management system 130 may be configured to determine a workload burnout risk score for each caregiver 102. As explained above, the workload burnout risk score may indicate the likelihood that the caregiver will burn out given recent workload conditions experienced on the job, such as the number of patients 104 or the medical complexity of those patients under the caregiver's care. The workload burnout risk score may be calculated based on such workload conditions experienced on a shift, over a number of shifts, or over some other period of time (e.g., a month or year). Burnout risk management system 130 may use a plurality of workload factors, discussed below, to determine the workload burnout risk score for each caregiver. The workload factors may correspond to a respective type of workload condition the caregiver experiences on the job. In one embodiment, burnout risk management system 130 may calculate a sub-score value for each workload factor and combine the sub-score values—e.g., adding the sub-scores directly or weighting each sub-score and adding the weighted sub-scores—to determine the overall workload burnout risk score based on its constituents.

For example, one workload factor may be a patient workload factor based on the number of patients 104 for which the caregiver is responsible. It will be appreciated that the more patients 104 under a caregiver's care, the greater the caregiver's workload and risk of burnout. In one embodiment, burnout risk management system 130 may determine a sub-score (e.g., a value on a range of 1-5, with 1 being the lowest risk/least patients and 5 being the highest risk/most patients) for the patient workload factor based on data received from caregiver communication system 116 and/or EHR system 112 indicating the number of patients under the caregiver's care. For example, caregiver communication system 116 may provide patient status information for the caregiver's shifts listing, among other things, the number of patients under the caregiver's care during the shifts. Alternatively, EHR system 112 may provide EHRs for the caregiver's patients, and burnout risk management system 130 may analyze the EHRs to determine the number of patients under the caregiver's care during the caregiver's shifts. Burnout risk management system 130 may, for example, determine a sub-score for the patient workload factor based on the total and/or average number of patients 104 under the caregiver's care per shift or over a number of shifts.

A second workload factor may be a patient medical complexity workload factor. It will be appreciated that the medical complexity of a caregiver's patients 104 may contribute to the burnout risk for that caregiver. For example, if a caregiver has patients 104 with a high risk of infection, the caregiver may need to dress/undress with a gown, cap, mask and/or take other protective measures each time the caregiver enters/leaves the patient's room, adding to the caregiver's workload and risk of burnout. Similarly, patients on dialysis or other palliative care require extra care that contributes significantly to the caregiver's workload, increasing stress and risk of burnout.

In one embodiment, burnout risk management system 130 may calculate a sub-score (e.g., a value on a range of 1-5, with 1 being the lowest risk/least medically complex and 5 being the highest risk/most medically complex) for the patient medical complexity workload factor. In one embodiment, burnout risk management system 103 use on EHR data from caregiver communication system 116 and/or from EHR system 112 for the caregiver's patients. For example, a patient's EHR may include data field indicating the medical complexity of the patent, which burnout risk management system 130 may use in this determination. Alternatively, burnout risk management system 130, using text-recognition capability, may parse EHRs to identify medical conditions suffered, or treatments undergone, by the patients. By comparing the identified conditions or treatments to a known database of conditions or treatments and their corresponding medical complexities, burnout risk management system 130 may determine the medical complexities of the caregiver's patients. The medical complexity for an individual patient may be determined, for example, as a binary value (e.g., complex or not complex) or as a value on a sliding scale (e.g., from 1-5, with 1 being simple and 5 being extremely complex). Depending on the implementation, for example, burnout risk management system 130 may determine a sub-score for the patient medical complexity workload factor based on the maximum complexity of any one of the caregiver's patients or the average medical complexity of all the caregiver's patients on a shift, over a number of shifts, or over another period of time (e.g., a month or a year).

Another workload factor may be a patient demand workload factor. It will be appreciated that some patients 104 demand more attention from their caregivers than others. For example, some patients 104, although under doctor's orders for bed rest, may frequently get out of bed 120 for various reasons, triggering a bed exit alarm to which the caregiver must respond. This adds to the caregiver's workload and risk of burnout. Accordingly, in one embodiment, burnout risk management system 130 may calculate a sub-score (e.g., a value on a range of 1-5, with 1 being the lowest risk/least demanding and 5 being the highest risk/most demanding) for the patient demand workload factor indicating the number of instances in which a caregiver's patients behave in a manner that demands attention and/or action from the caregiver on a shift, over a number of shifts, or over another period of time.

Burnout risk system 130 may determine instances in which patients demand caregiver attention in a number of ways. For example, burnout risk management system 130 may determine a number of bed exits by the caregiver's patients over a given period of time (e.g., the number of bed exits occurring within a single 12-hour shift). To do so, burnout risk management system 130 may receive data (e.g., bed exit records/notifications) over network(s) 132 from bed 120 and/or load sensor 136 and determine the total or average number of bed exits by the caregiver's patients.

As another example, demanding patients may frequently use caregiver call system 114 and/or caregiver communication system 116 to call and/or make requests of their caregivers. It will be appreciated that the more patient calls and/or requests to which the caregiver must respond in a shift or over a number of shifts, the greater the caregiver's workload and risk of burnout. To determine the number of instances in which patents of the caregiver call or make a request of the caregiver, burnout risk management system 130 may receive call and/or request record data from caregiver call system 114 and/or caregiver communication system 116 over network(s) 132. Based on this data, burnout risk management system 130 may determine the total or average number of times the caregiver's patients called and/or made requests of the caregiver in a shift or over a number of shifts.

As another example, caregivers 102 may be required to spend more time with demanding patients 104 (e.g., in the patient's room) for various reasons. This may increase the caregiver's workload and risk of burnout, as it can be exhausting and frustrating to spend too much time with a particular patient. Accordingly, in some embodiments, burnout risk management system 130 may determine the number of instances in which the caregiver spends time with a patient (e.g., visits the patient's room), spends more than a certain amount of time with a patient, etc. on a shift, over a number of shifts, or over another period of time. To do so, burnout risk management system 130 may receive location information for the caregiver from tracking system 118 and/or from wearable device 122. Based on the received location information of the caregiver as compared to a known layout of the clinical environment 100 (e.g., stored map information), burnout risk management system 130 may determine instances in which the caregiver spends time (or more than a certain amount of time) in the rooms of the caregiver's patients. As another example, burnout risk management system 130 may further receive location information of the caregiver's patients (e.g., an identification of which room the caregiver is in) from tracking system 118 and determine instances in which the caregiver is within a certain distance of any one of the caregiver's patients or within a certain distance of the caregiver's patients for more than a certain amount of time. As another example, caregiver communication system 116 may provide location status information for the caregiver and the caregiver's patients—such as an identification of which room(s) the caregiver and patients are located in—and determine that the caregiver is with a patient when their respective location statuses indicate that they are in the same room.

Another workload factor may be a patient admission/discharge workload factor. Admitting and discharging patients 104 requires significant administrative work (e.g., paperwork)—in addition to the usual medical care work—of their caregivers 102. Thus, it will be appreciated that a caregiver's workload and risk of burnout may increase with the number of admissions and/or discharges the caregiver must complete in a shift or over a number of shifts. Accordingly, in some examples, burnout risk management system 130 may calculate a sub-score (e.g., a value on a range of 1-5, with 1 being the lowest risk/least admissions/discharges and 5 being the highest risk/most admissions/discharges) for the patient admission/discharge workload factor based on EHR data for the caregiver's patients received from EHR system 112 and/or caregiver communication system 116 over network(s) 132. Based on this data, burnout risk management system 130 may determine the total or average number of patients the caregiver admitted and/or discharged on a shift, over a number of shifts, or over another period of time (e.g., a month or a year). And based on the number of admissions/discharges, burnout risk management system 130 may calculate a sub-score for the patient admission/discharge workload factor.

Another workload factor may be a medical procedure length workload factor. It will be appreciated that the more patients 104 a caregiver 102 has undergoing medical procedures (e.g., surgery) in a shift or over a number of shifts, the greater the caregiver's workload and risk of burnout because medical procedures can be intense, stressful, tedious, and time-consuming. Additionally, the nature of medical procedures often make needed breaks (e.g., bathroom, food, water breaks) impractical, if not impossible. Accordingly, burnout risk management system 130 calculate a sub-score (e.g., a value on a range of 1-5, with 1 being the lowest risk/least procedure time and 5 being the highest risk/most procedure time) for the medical procedure length workload factor based on data from EHR system 112, caregiver communication system 116, and/or tracking system 118. For example, burnout risk management system 130 may determine the number of instances in which the caregiver is involved in a medical procedure and/or the total or average amount of time the caregiver is involved in medical procedures with patients 104 in a shift, over a number of shifts, or over a period of time. And, based on the number of medical procedures and/or amount of time involved therein, burnout risk management system 130 may calculate a sub-score for the medical procedure length workload factor.

To determine whether a caregiver is involved in a medical procedure, for example, caregiver communication system 116 may provide location status information for the caregiver and the caregiver's patients. If the location statuses indicate that the caregiver is in a medical procedure room (e.g., operating room) with a patient, burnout risk management system 130 may determine that the caregiver is involved in a medical procedure. Alternatively, by comparing location information for the caregiver received from wearable device 122 to a known layout of the clinical facility (e.g., stored map information), burnout risk management system 130 may determine whether the caregiver is located in a medical procedure room and thus involved in a medical procedure. Burnout risk management system 130 may determine the medical procedure length workload factor based further in part on the determined time the caregiver was involved in medical procedures.

It will be appreciated that, due to the nature of medical procedures such as surgery, a caregiver's risk of burnout may increase at a greater rate for each incremental additional amount of time, or number of times, the caregiver is involved in medical procedures. This is because it becomes increasingly more difficult to tolerate a tedious task as a person reaches his or her limit. Thus, for example, a 6-hour surgical procedure may be more than twice as difficult to undertake than a 3-hour surgical procedure. Accordingly, in some examples, the medical procedure length workload factor may have a nonlinear relationship (e.g., an exponential) to the amount of time involved in medical procedures.

Another exemplary workload factor may be a caregiver urgency workload factor. It will be appreciated that, the more time (or number of times) a caregiver 102 spends urgently running or rushing throughout clinical environment 100, the greater the caregiver's workload and risk of burnout. Accordingly, burnout risk management system 130 may calculate a sub-score (e.g., a value on a range of 1-5, with 1 being the lowest risk/lowest urgency and 5 being the highest risk/most urgency) for the caregiver urgency workload factor based on the total or average amount of time the caregiver spends walking or running at a speed greater than a threshold speed during a shift, over a number of shifts, or over another period of time (e.g., a week, a month, or a year). To determine whether the caregiver is running/rushing, risk system 130 may use location data for the caregiver received from wearable device 122 and/or from tracking system 118 to determine the speed at which the caregiver is moving. If the caregiver is moving at a speed greater than a threshold, burnout risk management system 130 may determine that the caregiver is running/rushing.

Another exemplary workload factor may be a staffing workload factor. It will be appreciated that the amount and/or type of help or support available to a caregiver 102 on a shift may impact the caregiver's workload, stress level, and thus risk of burnout. For example, in general, having more clinical staff on a shift with the caregiver may decrease the caregiver's workload in the sense that the same total workload is spread across more people. Additionally, the experience level and/or knowledge of the clinical staff may impact the extent to which a caregiver can rely on that support. For example, on a given shift, the caregiver may have a greater workload if working with float staff (i.e., temporary or inexperienced staff) than if working with the caregiver's usual, permanent team.

Accordingly, in some examples, burnout risk managements system 130 may calculate a sub-score (e.g., a value on a range of 1-5, with 1 being the lowest risk/most help and 5 being the highest risk/least help) for the staffing workload factor based in part on the total or average number of clinical staff working with the caregiver in a shift or over a number of shifts. For example, the sub-score may be reduced as the number of clinical staff increases. Additionally, burnout risk management system 130 may determine the staffing workload factor sub-score based further on the experience level of clinical staff working with the caregiver. For example, the factor may be increased as the ratio of inexperienced staff (e.g., float or temporary) to total clinical staff increases. To determine the number and experience level of clinical staff working with the caregiver on a shift, burnout risk management system 130 may use data received from caregiver communication system 116 and/or tracking system 118.

For example, burnout risk management system 130 may receive location information for the caregiver and the other clinical staff on the shift from wearable device(s) 122 and/or from tracking system 118. Based on the location of the clinical staff being within a certain range of the location of the caregiver during a shift, for example, the number of clinical staff supporting the caregiver may be determined. As another example, based on the caregiver and other clinical staff being logged in to caregiver communication system 116 and therefore at work, caregiver communication system 116 may provide to burnout risk management system 130 status information and/or location information indicating the clinical staff members currently working together on a shift. Additionally, based on the identity of the clinical staff, burnout risk management system 130 may determine whether each clinical staff is float staff or permanent staff (e.g., inexperienced or experienced). For example, burnout risk management system 130 may compare the identity of the clinical support staff to a known roster of clinical employees and, based on their respective job titles/functions, determine the experience level of the clinical staff members. As another example, caregiver communication system 116 may provide the identities and/or job titles/functions of each clinical staff member working with the caregiver on a shift, which caregiver burnout system may use to make the experience level-determination. Based on the average experience level of the clinical staff working with the caregiver on a shift, over a number of shifts, or over another period time, burnout risk management system 130 may increase/decrease the sub-score for the staffing workload factor.

Another exemplary workload factor may be a break workload factor. It will be appreciated that a caregiver's workload and risk of burnout increases if the caregiver has insufficient breaks for food, water, bathroom, etc. Accordingly, in some examples, burnout risk management system 130 may calculate a sub-score (e.g., a value on a range of 1-5, with 1 being the lowest risk/most break time and 5 being the highest risk/least break time) for the break workload factor based on the total or average amount of time the caregiver spends on break in a shift, over a number of shifts, or over another period of time. To determine whether the caregiver is on break, burnout risk management system 130 may receive location data for the caregiver from wearable device 122 and/or from tracking system 118. By comparing the location of the caregiver to known locations of break areas, kitchens, bathrooms, water fountains, etc. (e.g., based on stored map information for the clinical environment 100), burnout risk management system 130 may determine that the caregiver is on break. Alternatively, break areas, bathrooms, water fountains, and/or other objects and/or areas caregivers use on breaks may be tagged in tracking system 118 as discussed above, and burnout risk management system 130 may receive location information for these objects and/or areas used on breaks from tracking system 118. Based on the location of the caregiver being within a certain distance of the locations of such objects and/or areas used on breaks, burnout risk management system 130 may determine that the caregiver is on break. As another example, the location status information received from caregiver communication system 116 may identify the particular room/area in which the caregiver is located, and burnout risk management system 130 may determine whether the caregiver is on break based on the type or purpose of room/area. For example, burnout risk management system 130 may use a stored list of all rooms/areas in the clinical environment 100 that identifies the corresponding use purpose(s) for each area/room to make this determination.

Another exemplary workload factor may be a shift length/overtime workload factor. It will be appreciated that a caregiver's workload and risk of burnout increases with shift length, the amount of overtime, or the total amount of hours a caregiver 102 works over a given period of time. Accordingly, in some examples, burnout risk management system 130 may calculate a sub-score (e.g., a value on a range of 1-5, with 1 being the lowest risk/least overtime and 5 being the highest risk/most overtime) for the shift length/overtime workload factor based on the total or average length of the caregiver's shift(s), the total or average amount of overtime the caregiver works over a period of time (e.g., a month or a year), and/or the total amount of time the caregiver works over a period of time (e.g., a month). Burnout risk management system 130 determine whether the caregiver is working in a variety of ways. For example, burnout risk management system 130 may determine whether the caregiver is logged into tracking system 118 based on status information received from tracking system 118. Alternatively, burnout risk management system 130 may receive location information for the caregiver from tracking system 118 and determine that the caregiver is working for the time that tracking system 118 has a known location for the caregiver or that the caregiver is located within the clinical environment. Alternatively, burnout risk management system 130 may receive location information from the caregiver's wearable device 122 and determine that the caregiver is working if the location is within the clinical facility.

Another exemplary workload factor may be a visitor involvement workload factor. It will be appreciated that a caregiver's workload and risk of burnout increases to the extent that the caregiver not only has to work with the patient, but with family, friends, or other visitors of the patient. For example, sometimes caregivers must do extra administrative work, such as phone calls and paperwork, to accommodate visitors. Alternatively, in some circumstances, friends or family may actually give nursing care (e.g., feeding, changing, or ambulating the patient) or otherwise help the caregiver(s). Accordingly, in some examples, burnout risk management system 130 may calculate a sub-score (e.g., a value on a range of −5 to +5, with −5 representing the lowest risk/most positive visitor interaction, 0 being neutral, and 5 being the highest risk/most positive visitor interaction; a value on a range of 1-5 etc.) for the visitor involvement workload factor based on the extent to which a caregiver 102 interacts with visitors of any patients and calculate the visitor involvement factor based thereon. For example, the visitor involvement factor may be determined based on the total or average number of visitors interacted with on the caregiver's shift(s), the total or average amount of the time the caregiver spends interacting with visitors on the caregiver's shift(s), etc. To determine whether the caregiver interacts with visitors, for example, visitors may be given bracelets with tags for tracking system 118 used to determine the location, identity, and/or patient associated with the visitor, and this information may be communicated to burnout risk management system 130. For example, based on the location of the visitor being within a certain range of the caregiver and the identity information for the visitor being associated with one of that caregiver's patients, burnout risk management system 130 may determine that the caregiver is interacting with the visitor. Alternatively, burnout risk management system 130 may use a text recognition function to parse EHR data for the patient received from EHR system 112 to determine whether the caregiver interacted with visitors.

Alternatively, burnout risk management system 130 may receive message records associated with the caregiver's patients from caregiver communication system 116. Based on the message records indicating messages from the caregiver to a visitor of the patient and/or from a visitor to the caregiver, burnout risk management system 130 may determine that the caregiver is interacting with a visitor.

In one embodiment, burnout risk management system 130 may calculate the workload burnout risk score as follows:

Workload Burnout Risk Score=A*factor1+B*factor2+ . . . +Z*factorX,

wherein factors 1 through X include the calculated workload factors discussed above, such as the patient workload factor, patient medical complexity workload factor, patient demand workload factor, patient admission/discharge workload factor, medical procedure length workload factor, caregiver urgency workload factor, staffing workload factor, and shift length/overtime workload factor. Additionally, the values of A through Z may be weighting coefficients applied to each of the workload factors to calculate the workload burnout risk score. The values for the coefficients may be chosen accordingly by one with knowledgeable of the clinical environment 100 and of the relative impact of the various workload factors on the risk of caregiver burnout. For example, based on knowledge that understaffing increases workload and contributes to burnout more significantly than visitor involvement, the coefficient for the staffing workload factor may be made greater than the coefficient for the visitor involvement workload factor. These coefficients may also have weighting that varies based on the caregiver. For example, a caregiver with more medical training or experience may be less impacted by medical complexity than a caregiver with less training or experience. The factor for medical complexity may have a smaller weighting coefficient for the well-trained caregiver and a larger weighting factor for the caregiver with less training.

Emotional Burnout Risk Score

Based on the data received from the clinical data sources over communication network(s) 132, burnout risk management system 130 may be configured to determine an emotional burnout risk score for each caregiver 102. The emotional burnout risk score may indicate the likelihood of a caregiver burning out based on the amount of emotional labor and/or stress a caregiver 102 encounters over a shift, a number of shifts, or some other period of time (e.g., a month or year). Burnout risk management system 130 may use a plurality of emotional burnout risk factors, examples of which are discussed below, to determine the emotional burnout risk score for each caregiver 102. The emotional burnout risk factors may correspond to a respective type of emotional condition/event the caregiver experiences on the job. In one embodiment, burnout risk management system 130 may calculate a sub-score value for each emotional burnout risk factor and combine the sub-score values—e.g., adding the sub-scores directly or weighting each sub-score and adding the weighted sub-scores—to determine the overall emotional burnout risk score based on its constituents.

One exemplary emotional burnout risk factor may be a violent events factor. It will be appreciated that caregivers 102 sometimes encounter aggressive, angry, confused, or otherwise disturbed patients 104 that commit violent acts (e.g., screaming, assaulting, or throwing objects) against their caregivers. For example, a psychiatric patient may have a psychotic break and, in a state of confusion and fear, attack his or her caregiver or throw a television across the room. Such violent events can be very traumatic, producing long-lasting effects on caregivers and contributing to risk of burnout.

Accordingly, in some examples, burnout risk management system 130 may be configured to calculate a sub-score (e.g., a value on a range of 1-5, with 1 being the lowest risk/least violence and 5 being the highest risk/most violence) for the violent events factor based on the total or average number of violent events a caregiver 102 encounters on a shift, over a number of shifts, or over another period of time. Burnout risk management system 130 may detect violent events in a number of ways. For example, burnout risk management system 130 may analyze video data received from camera(s) 108 to identify altercations between the caregiver and patients or between caregivers. In some exemplary implementations, burnout risk management system 130 may apply known image processing techniques to detect events in which identified objects in the video data, corresponding caregiver and/or patients, aggressively move toward and/or contact others. Burnout risk management system 130 may identify the caregiver involved in the violent event, for example, based on information indicating that the camera(s) 108 from which the video data was received is associated with a room/area associated with a patient of the caregiver, on face and/or body recognition techniques applied to the video data, or on other techniques. Alternatively, or additionally, burnout risk management system 130 may use location information received from caregiver communication system 116, tracking system 118, and/or wearable device 122 to determine that the caregiver was located in the same room/area as the camera(s) 108 that captured a detected violent event.

Caregiver burnout system 130 may similarly use audio data received from microphone(s) 110 to detect violent events involving the caregiver. For example, using voice recognition software, burnout risk management system 130 may analyze the audio data to detect episodes of persons speaking in raised voices (e.g., yelling or screaming); objects crashing, breaking, or being thrown; etc. And using knowledge of the location of the microphone(s) 110 that captured the audio of the violent event and/or location data for the caregiver received from caregiver communication system 116, tracking system 118, and/or wearable device 122, burnout risk management system 130 may determine that the caregiver was involved in the violent event. Alternatively, burnout risk management system 130 may apply known voice recognition techniques to identify the caregiver and/or patient involved in a violent event.

As another example, burnout risk management system 130 may detect violent events based on EHR information received from EHR system 112. For example, using text-recognition software, burnout risk management system 130 may parse the EHR information of the caregiver's patients to determine whether any violent events have been noted.

Another exemplary emotional burnout risk factor may be a negative interaction factor. It will be appreciated that negative interactions with the family, other visitors of a patient 104, or other caregivers or clinical staff, can be emotionally difficult for a caregiver 102, contributing to the risk of burnout. For example, family members may argue with the caregiver about the plan for care, visiting hours, or other issues. Accordingly, in some examples, burnout risk management system 130 may be configured to calculate a sub-score (e.g., a value on a range of 1-5, with 1 being the lowest risk/least negative interactions and 5 being the highest risk/most negative interactions) for the negative interaction factor based on the total or average number of negative interactions a caregiver 102 in which the caregiver is involved on a shift, over a number of shifts, or over another period of time (e.g., a month or a year).

Burnout risk management system 130 may detect negative interactions in a number of ways. For example, burnout risk management system 130 may analyze video data received from camera(s) 108 to identify negative interactions between the caregiver and family members. In some exemplary implementations, the video data may be analyzed to identify events in which the caregiver and/or family members move aggressively move toward and/or contact one another, speak to one another in raised voices, etc. Burnout risk management system 130 may identify the caregiver involved in the negative interaction, for example, based on information indicating that the camera(s) 108 from which the video data was received is associated with a room/area associated with a patient of the caregiver. Alternatively, or additionally, burnout risk management system 130 may use location information received from caregiver communication system 116, tracking system 118, and/or wearable device 122 to determine that the caregiver was located in the same room/area as the camera(s) 108 that captured the negative interaction. In another example, burnout risk management system 130 may use face and/or body recognition techniques to identify the caregiver and/or patient involved in the negative interaction.

Burnout risk management system 130 may similarly use audio data received from microphone(s) 110 or from caregiver communication system 116 to detect negative interactions involving the caregiver. For example, using voice recognition software, burnout risk management system 130 may analyze the audio data to detect episodes of persons speaking in raised voices (e.g., yelling or screaming); objects crashing, breaking, or being thrown; etc. And using knowledge of the location of the microphone(s) 110 that captured the audio of the negative interaction and/or location data for the caregiver and/received from caregiver communication system 116, tracking system 118, and/or wearable device 122, burnout risk management system 130 may determine that the caregiver was involved in the negative interaction. Additionally, since family visitors may be given a tag for tracking system 118, burnout risk management system 130 may determine that the interaction involved family of a patient of the caregiver based on the identification and/or location information of that tag received from tracking system 118.

As another example, burnout risk management system 130 may detect negative interactions based on EHR information received from EHR system 112. For example, using text-recognition software, burnout risk management system 130 may parse the EHR information of the caregiver's patients to determine whether any negative interactions are noted in the patients' EHRs. Alternatively, burnout risk management system 130 may receive message records associated with the caregiver's patients from caregiver communication system 116. Based on the message records indicating messages to/from a family member of the caregiver's patients, burnout risk management system 130 may determine that the caregiver is interacting with a visitor. Additionally, burnout risk management system 130 may parse the content of the message records to determine the subject matter thereof and whether it relates to a negative interaction between the caregiver and a family member of the patient.

Another exemplary emotional burnout risk factor may be a patient death factor. It will be appreciated that the death of a patient 104 can be very emotionally difficult for a caregiver 102 and contribute to the risk of burnout, particularly if the caregiver had a close relationship with the patient. Accordingly, in some embodiments, burnout risk management system 130 may be configured to calculate a sub-score (e.g., a value on a range of 1-5, with 1 being the lowest risk/least deaths and 5 being the highest risk/most deaths) for the patient death factor based on the total or average number deaths of patients under the care of the caregiver on a shift, over a number of shifts, or over a longer period of time (e.g., a month or year). To determine whether a patient of the caregiver died, burnout risk management system 130 may analyze EHR information received from EHR system 112. For example, using text-recognition software, burnout risk management system 130 may parse the EHR information of the caregiver's patients to whether any deaths have been noted. Another consideration for assigning the score for a patient death could be how much time the caregiver had spent with the patient, using the techniques discussed above to determine the amount of time the caregiver has spent with a patient who has died (e.g., location information a caregiver communication system 116, a personnel/asset tracking system 118, or another clinical data source). The score could be higher for a caregiver that spent a greater cumulative time with a patient and lower for a caregiver that had less interaction with the patient.

In one embodiment, burnout risk management system 130 may calculate the emotional burnout risk score as follows:

Emotional burnout risk Score=A*factor1+B*factor2+ . . . +Z*factorX,

wherein factors 1 through X include the calculated emotional burnout factors discussed above, such as the violent events factor, negative interaction factor, and patient death factor. Additionally, the values of A through Z may be weighting coefficients applied to each of the emotional factors to calculate the emotional burnout risk score. The values for the coefficients may be chosen accordingly by one with knowledgeable of the clinical environment 100 and of the relative impact of the various emotional factors on the risk of caregiver burnout. For example, based on knowledge that a violent event tends to be the most emotionally-taxing emotional for a caregiver, the coefficient for the violent events factor may be made greater than the coefficients for the remaining emotional factors. These coefficients may also have weighting that varies based on the caregiver. For example, a caregiver with a certain personality trait may be more affected by a patient death than a caregiver with a different trait. The factor for patient death may have a greater weighting coefficient for the caregiver predisposed to forming tighter emotional bonds with patients and lower for a caregiver without that trait.

Physical Burnout Risk Score

Based on the data received from the clinical data sources over communication network(s) 132, burnout risk management system 130 may be configured to determine a physical burnout risk score for each caregiver 102. The physical burnout risk score may indicate the likelihood of the caregiver burning out given the amount of physically-demanding work a caregiver 102 performs over a shift, a number of shifts, or some period of time (e.g., a month or year). Burnout risk management system 130 may use a plurality of physical factors to determine the physical burnout risk score for each caregiver 102, each corresponding to a respective type of physically-demanding work the caregiver performs on the job. In one embodiment, burnout risk management system 130 may calculate a sub-score value for each physical burnout risk factor and combine the sub-score values—e.g., adding the sub-scores directly or weighting each sub-score and adding the weighted sub-scores—to determine the overall physical burnout risk score based on its constituents.

One exemplary physical factor may be a distance walked factor. It will be appreciated that the further a caregiver walks, or the more steps that a caregiver takes during a shift, over a number of shifts, and/or over a longer period of time (e.g., a month or a year), the more physically-demanding caregiver's the work. For example, sometimes caregivers must walk back and forth between different departments of a clinical facility, separated by a significant distance, multiple times per shift, which can be physically exhausting and increase risk of burnout. Furthermore, pushing an occupied bed, stretcher, or other wheeled device can exert even more physical stress to the body than walking alone.

Accordingly, in some examples, burnout risk management system 130 may be configured to calculate a sub-score (e.g., a value on a range of 1-5, with 1 being the lowest risk/least physically-demanding and 5 being the highest risk/most physically-demanding) for the distance walked factor based on the distance a caregiver 102 walks on a shift or over a number of shifts. Burnout risk management system 130 may determine the distance in a number of ways, such as based on distance-walked and/or step information received from wearable device 122, location information received from tracking system 118, and/or location information received from caregiver communication system 116. The greater the distance walked in the period of time, the greater the distance walked factor. Additionally, tracking system 118 can also be used to identify whether a bed, stretcher, and/or patient are present with the caregiver to indicate the caregiver is transporting the patient. Distance walked while pushing a patient and piece of equipment, can have an even greater influence on increasing the distance walked factor than walking alone.

Another exemplary physical factor may be a time standing factor. Standing for a long time can be physically demanding and contribute to the risk of caregiver burnout. For example, caregivers involved in medical procedures (e.g., surgery) often must remain on their feet for hours at a time without the opportunity to sit and rest. Accordingly, in some examples, burnout risk management system 130 may be configured to calculate a sub-score (e.g., a value on a range of 1-5, with 1 being the lowest risk/least standing and 5 being the highest risk/most standing) for distance walked factor based on the amount of time a caregiver 102 spends standing on a shift, over a number of shifts, or over another period of time (e.g., a week or month). As another example, burnout risk management system 130 may determine the sub-score based on the number of shifts in which the caregiver predominately stands, or stands for more than a certain amount of time. Additionally, in some cases, the number of shifts or amount of time the caregiver sits may be used to reduce the sub-score for the time standing factor. That is, shifts/days spent predominately standing may increase the sub-score while shifts/days spent predominately sitting may reduce the sub-score.

Burnout risk management system 130 may determine the amount of time standing in a number of ways, such as based on distance-walked, step, and/or acceleration information received from wearable device 122, location information received from tracking system 118, and/or location information received from caregiver communication system 116. The greater the time spent standing, the greater the sub-score for the time standing factor. Additionally, as discussed above a plurality of wearable devices 122 may be used to estimate the posture of a caregiver and thereby determine whether the caregiver is standing or sitting for purposes of the time standing factor.

Another exemplary physical factor may be a patient handling activity factor. Carrying, moving, lifting, pushing, or otherwise manipulating an immobile patient can be physically demanding and contribute to the risk of caregiver burnout if done frequently. Accordingly, in some examples, burnout risk management system 130 may be configured to calculate a sub-score (e.g., a value on a range of 1-5, with 1 being the lowest risk/least handling and 5 being the highest risk/most handling) for the patient handling activity factor for a caregiver 102 based on the type of activity and the number of times (e.g., total or average) the caregiver physically handles a patient during a shift, over a number of shifts, or over another period of time (e.g., a week or month). Burnout risk management system 130 may determine instances in which the caregiver handles a patient in a number of ways. For example, if the caregiver is determined to be in the proximity of a patient and bed 120 and/or load sensor 136 detects the patient to be lifted off the bed, burnout risk management system 130 may determine that the caregiver handles a patient. Video 108 may also be used as a means for the burnout risk management system 130 to determine that the caregiver handles a patient. Based on a combination of bed 120, load sensor 136, and video 108; the type of patient handling task can be assumed. The contribution to the patient handling activity factor may be higher for a more strenuous task such as transferring a patient from bed to chair where the patient must be lifted against gravity, and lower for a task such as repositioning a patient in bed where the patient is slid across the mattress. The contribution to the patient handling activity factor may be higher based on EHR information received from EHR system 112 for patients that are more physically dependent or weigh more. The contribution of a patient handling task to the patient handling activity sub-score may be reduced or even ignored if patient lift system 128 is used. As an example, patient lift system 128 may be tagged in tracking system 118, and burnout risk management system 130 may determine that the caregiver is handling a patient safely when the received location information for patient lift system 128 is within a distance of that of the caregiver. Alternatively, burnout risk management system 130 may receive lift records from patient lift system 128 and determine from information in the records that the caregiver used lift system 128 to handle a patient. Alternatively, burnout risk management system 130 may analyze EHR information for the caregiver's patients, received from EHR system 112, to determine whether any patient-handling events are noted.

In one embodiment, burnout risk management system 130 may calculate the physical burnout risk score as follows:

Physical burnout risk Score=A*factor1+B*factor2+ . . . +Z*factorX,

wherein factors 1 through X include the calculated physical factors discussed above, such as the distance walked factor, time standing factor, and/or patient handling activity factor. Additionally, the values of A through Z may be weighting coefficients applied to each of the physical factors to calculate the emotional burnout risk score. The values for the coefficients may be chosen accordingly by one with knowledgeable of the clinical environment 100 and of the relative impact of the various physical factors on the risk of caregiver burnout. For example, if moving, lifting, or otherwise manipulating a patient is considered the most physically demanding task of a caregiver 102, its coefficient may be made greater than the others. These coefficients may also have weighting that varies based on the caregiver. For example, a caregiver who is older, shorter, lighter, and/or female may be more affected by a manual patient handling activity than a caregiver who is younger, taller, heavier, and/or male. The factor for patient handling activity may have a greater weighting coefficient for the older, shorter, lighter, and/or female caregiver than a caregiver with different traits.

In one embodiment, burnout risk management system 130 may calculate the total burnout risk score as follows:

Total Burnout Risk Score=A*workload burnout risk score+B*emotional burnout risk score+C*physical burnout risk score,

where A through C are respective coefficients applied to the workload, emotional, and physical burnout risk scores discussed above to calculate the total burnout risk score. The values for the coefficients may be chosen accordingly, such as based on knowledge of the relative contribution to workload, emotional labor, and physical labor to caregiver burnout. In some embodiments, users may benefit from understanding where the score and/or sub-scores come from so that they better trust the scores. Accordingly, one embodiment may normalize the separate sub-scores and present them as a scale that sums to 100. For example, based on the relative contributions to burnout, the workload sub-score may be X out of 50, the emotional sub-score may be Y out of 30, and the physical sub-score may be Z out of 20. Thus, the total burnout risk score may be 100, and the different importance of the respective sub-scores may be weighted accordingly to make up the total score of 100.

Burnout risk management system 130 may be configured to send a report based on the calculated total burnout risk score. For example, if burnout risk management system 130 determines that the total burnout risk score for any caregiver 102 is greater than a threshold, burnout risk management system 130 may send a report message to administrator 106, notifying administrator of the risk of potential burnout of the caregiver. The threshold for notification may be fixed across an entire facility, or it could be variable based on the unit or even the individual caregiver. The report message may include, for example, the total burnout risk score, the workload burnout risk score, the emotional burnout risk score, and/or the physical burnout risk score for the caregiver. Additionally, the report may be sent by email, text message, instant messaging (e.g., via a messaging platform used by the clinical facility or via caregiver communication system 116). In this manner, administrator 106 may take preventative measures in advance of the caregiver actually burning out.

Burnout risk management system 130 may further provide reporting on the burnout risk scores at the enterprise/facility level for clinical environment 100. Such reports may show the scores for individual caregivers in each unit or department of the facility (e.g., labor and delivery, surgery, ICU, etc.) Based on this information, administration may make changes to staffing across the different units or departments, or take other measures, to mitigate or reduce risk of burnout in the units or departments with the highest risk.

It will be appreciated that the calculation of the burnout risk score (and its constituent sub-scores) and reporting of the same, discussed above, may allow administrator 106 to proactively address potential caregiver burnout before it occurs. For example, the affected caregivers may be given time off, given different schedule, moved to a different department, given more support (e.g., additional staff) on their shifts, scheduled for an intervention/treatment, etc.

In addition to preventatively determining and warning/alerting of the risk of potential burnout through the total burnout risk score discussed above, burnout risk management system 130 may be configured to determine an actual burnout score indicating whether the caregiver has burned out and/or is exhibiting signs of burnout. For example, some caregivers may be at risk of burnout but have not yet burned out or begun to show signs of it. But other caregivers may have progressed beyond mere risk of burnout, into a current state of burnout or exhibiting signs of actual burnout. The burnout risk score, discussed above, may help to identify the former caregivers while the actual burnout score, discussed below, may help to identify the latter caregivers. The actual burnout score may be determined based on a variety of burnout factors.

One burnout factor may be a physiological burnout factor. For example, caregivers 102 who are burned out may be have elevated heart rate, excessive or insufficient heart rate variability (HRV), or excessive perspiration on account of being overworked or overly-stressed. HRV may be related to emotional arousal. High-frequency (HF) activity has been found to decrease under conditions of acute time pressure and emotional strain and elevated anxiety state. Additionally, HRV has been shown to be reduced in depressed individuals or those reporting to worry more. In individuals with post-traumatic stress disorder (PTSD), HRV and its HF component is reduced whilst the low-frequency (LF) component is elevated. Furthermore, PTSD patients demonstrated no LF or HF reactivity to recalling a traumatic event. Alternatively, the amount of time spent in stressful states (e.g., indicated by HR or HF) could also be used to determine a sub-score for the physiological burnout risk factor.

Burnout risk management system 130 may calculate a physiological burnout score based on physiological data received from the clinical data sources. For example, heart rate data may be received from wearable device 122. Based on the received heart rate data, burnout risk management system 130 may determine the total or average amount of time, or the total or average number of times, the caregiver's heart rate is elevated and/or exhibiting HRV during a shift, over a number of shifts, or over another time period (e.g., a week or a month). And based on the amount and/or number of times the caregiver's heart rate is elevated or in an HRV state, burnout risk management system 130 may calculate the physiological burnout score.

As another example, caregivers exhibiting signs of burnout may perspire more than normal or more than those who are not exhibiting signs of burnout. Accordingly, in some embodiments, skin conductivity data indicative of the amount the caregiver perspires may be received from wearable device 122. Based on the received skin conductivity data, burnout risk management system 130 may determine the total or average amount of time, or the total or average number of times, the caregiver's skin conductivity is greater than a threshold amount. And burnout risk management system 130 may calculate a value for the physiological burnout score based further on the amount and/or number of times the caregiver has elevated skin conductivity.

Another exemplary burnout factor may be an absentee/responsiveness burnout factor. For example, a caregiver 102 who is burned out may miss days of work, spend less time than normal with patients 104, not respond promptly to patient calls/requests, leave the caregiver's designated work area(s) more or less often than normal, etc. Burnout risk management system 130 may determine an absentee/responsiveness burnout score based on data received from the clinical data sources. For example, burnout risk management system 130 may receive location and/or status data for the caregiver received from tracking system 118, caregiver communication system 116, and/or wearable device 122. Based on the location/status data for the caregiver in comparison to a stored work schedule for the caregiver—burnout risk management system 130 may identify instances in which the caregiver is absent/tardy from/to work or otherwise has reduced responsiveness.

Additionally, by comparing the received location/status data for the caregiver to historical location/status data for the caregiver, burnout risk management system 130 may determine instances of reduced responsiveness in which the caregiver spends less time than historically normal (e.g., average) with patients or leaves the caregiver's designated work area more or less often than historically normal (e.g., average). Burnout risk management system 130 may determine further instances of absenteeism and/or reduced responsiveness, for example, based on patient requests/calls made using caregiver call system 114 and/or caregiver communication system 116 to which the caregiver does not respond within a historically normal amount of time. Based on the number of instances of absenteeism or reduced responsiveness in a shift, a number of shifts, or another period of time (e.g., a month or a year), burnout risk management system 130 may calculate a value for the absentee/responsiveness burnout score.

In one embodiment, burnout risk management system 130 may calculate the total actual burnout score as follows:

Actual Burnout Score=A*physiological burnout score+B*absentee/responsiveness burnout score,

where A and B are respective coefficients applied to the value for the physiological burnout score and the absentee/responsiveness burnout score discussed above to calculate the burnout score. The values for the coefficients may be chosen accordingly, e.g., by administrator 105.

Burnout risk management system 130 may be configured to send a report based on the calculated burnout score. For example, if burnout risk management system 130 determines that the burnout score for any caregiver 102 is greater than a threshold, burnout risk management system 130 may send a report message to administrator 106, notifying administrator of that the caregiver is exhibiting signs of burnout. The threshold for notification may be fixed across an entire facility, or it could be variable based on the unit or even the individual caregiver. The report message may include, for example, the burnout score, the physiological burnout score, and/or the absentee /responsiveness burnout score for the caregiver. Additionally, the report may be sent by email, text message, instant messaging (e.g., via a messaging platform used by the clinical facility or via caregiver communication system 116). In this manner, administrator 106 may take measures to mitigate the effects of burnout on the caregiver and/or the impact on the clinical facility more broadly.

FIG. 2 illustrates an exemplary configuration of burnout risk management system 130 to enable and/or perform the some or all the functionality discussed herein. Burnout risk management system 130 can be implemented as one or more server computers, a network element on a dedicated hardware, as a software instance running on a dedicated hardware, or as a virtualized function instantiated on an appropriate platform, such as a cloud infrastructure, and the like. It is to be understood in the context of this disclosure that burnout risk management system 130 can be implemented as a single device or as a plurality of devices with components and data distributed among them.

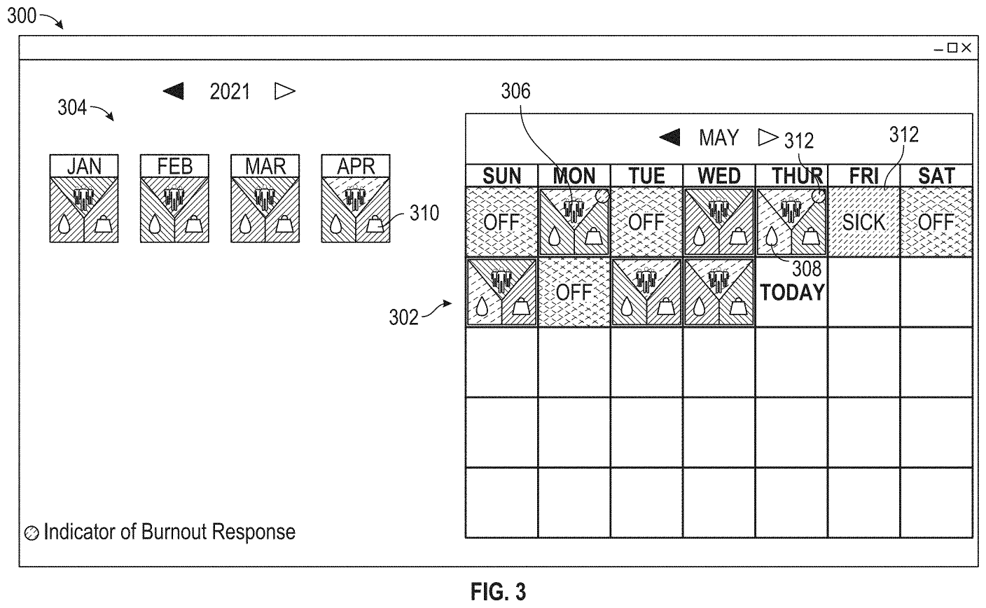

As illustrated, burnout risk management system 130 may comprise a memory 202. In various embodiments, memory 202 is volatile (including a component such as Random Access Memory (RAM)), non-volatile (including a component such as Read Only Memory (ROM), flash memory, etc.) or some combination of the two. Memory 202 may include various components, such as burnout risk score calculate engine 204, burnout score calculation engine 206, burnout reporting engine 208, and a burnout dashboard engine 210. Burnout risk score calculation engine 204, when executed, may be configured to perform the functionality discussed above to calculate the burnout risk score. Burnout score calculation engine 206, when executed, may be configured to perform the functionality discussed above to calculate the burnout score. Burnout reporting engine 208, when executed, may be configured to perform the reporting functions discussed above to report the burnout risk score, burnout score, and related information. Burnout dashboard engine 210, when executed, may be configured to provide the burnout dashboard graphical user interface (GUI) 300, discussed below with reference to FIG. 3. These engines may include methods, threads, processes, applications, software, computer code, or any other sort of executable instructions configured to perform the functionality disclosed herein when executed by one or more processor(s) 212. Elements 204-208 may also include associated files and databases.

Processor(s) 212 may a Central Processing Unit (CPU), a Graphics Processing Unit (GPU), or both CPU and GPU, or other processing unit or component known in the art configured to process data and execute computer program instructions.

Burnout risk management system 130 may include additional data storage devices (removable and/or non-removable) such as, for example, magnetic disks, optical disks, or tape. Such additional storage is illustrated in FIG. 2 by removable storage 214 and non-removable storage 216. Tangible computer-readable media can include volatile and nonvolatile, removable and non-removable media implemented in any method or technology for storage of information, such as computer readable instructions, data structures, program modules, or other data. Memory 202, removable storage 214, and non-removable storage 216 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 1000. Any such tangible computer-readable media can be part of burnout risk management system 130.

Burnout risk management system 130 also can include input device(s) 218, such as a keypad, a cursor control, a touch-sensitive display, voice input device, etc., and output device(s) 220 such as a display, speakers, printers, etc. These devices are well known in the art and need not be discussed at length here. In particular implementations, a user, such as administrator 106, can provide input to device(s) 218 via a user interface associated with input device(s) 218 and/or output device(s) 220.

As illustrated in FIG. 2, burnout risk management system 130 can also include one or more wired or wireless transceiver(s) 222. For example, transceiver(s) 222 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, transceiver(s) 222 can utilize Multiple-Input/Multiple-Output (MIMO) technology. Transceiver(s) 222 can include any sort of wireless transceivers capable of engaging in wireless, Radio Frequency (RF) communication. Transceiver(s) 222 can also include other wireless modems, such as a modem for engaging in Wi-Fi, WiMAX, Bluetooth, or infrared communication. In some implementations, transceiver(s) 222 can be used to communicate between various functions, components, modules, or the like, that are comprised in burnout risk management system 130.

As used herein, the term “communication network,” and its equivalents, may refer to at least one device and/or at least one interface over which data can be transmitted between endpoints. For instance, communication network(s) 132 may represent one or more communication interfaces traversing the communication network(s). Examples of communication networks include at least one wired interface (e.g., an ethernet interface, an optical cable interface, etc.) and/or at least one wireless interface (e.g., a BLUETOOTH interface, a WI-FI interface, a near-field communication (NFC) interface, a Long Term Evolution (LTE) interface, a New Radio (NR) interface, etc.). In some cases, data or other signals are transmitted between elements of FIG. 1 over a wide area network (WAN), such as the Internet. In some cases, the data include one or more data packets (e.g., Internet Protocol (IP) data packets), datagrams, or a combination thereof.

FIG. 3 illustrates an exemplary burnout dashboard graphical user interface (GUI) 300, consistent with the disclosed embodiments. In implementations, dashboard GUI 300 may be implemented by processors 212 (FIG. 2) executing computer program instructions of burnout dashboard element 210. Dashboard GUI 300 may be used by administrator 106, for example, to monitor caregiver(s) 102 and take measures in response to risk of burnout or potential actual burnout of caregiver(s) 102. In one embodiment, dashboard GUI 300 may provide a calendar visual representation of the burnout risk score for a selected caregiver 102, including the workload score, emotional score, and physical score, for each day/shift 304 and/or cumulatively for each month 304 worked by the selected caregiver.

In the example of FIG. 3, the workload score, emotional score, and physical score for each day/shift 302 and/or month 304 are represented by a workload burnout risk score icon 306 (group of patients), an emotional burnout risk score icon 308 (tear drop), and/or a physical burnout risk score icon 310 (anvil weight) respectively. Additionally, in the example, for each working day/shift 302 and/or month 304, icons 306-310 may be visually distinguished to indicate the value of the respective score on that working day/shift 302 and/or month 304. For example, the corresponding icon 306-310 may be respectively highlighted with a first color (e.g., green), a second color (e.g., yellow), and a third color (e.g., red) if the score for that day/shift 302 or month 304 falls within a first range (low burnout risk), a second range (medium burnout risk), and a third range (high risk).

Additionally, dashboard GUI 300 may include a burnout indicator icon 312 on the calendar to indicate a day/shift 302 in which the caregiver exhibited behavior suggestive of a response to burnout. In one embodiment, if burnout risk management system 130 calculates a value for the burnout score as discussed above that is greater than a threshold for a given day/shift 302, dashboard GUI 300 may include burnout indicator icon 312 on the calendar for that day/shift 302. In the example shown, the caregiver exhibited behavior suggestive of burnout (e.g., responsiveness/absenteeism or physiological response) on the first Monday, Tuesday, and Friday of September, and so those days/shifts 302 have been marked with burnout indicator icon 312. For example, on Monday the caregiver may have had elevated and/or variable heart rate episodes suggestive of burnout, on Thursday the caregiver may have had episodes of perspiration suggestive of burnout, and on Friday the caregiver called out sick.

In some embodiments, the calendar of GUI 300 may show respective values for the workload burnout risk score, emotional burnout risk score, and physical burnout risk score for each day/shift 302 and/or month 304 instead of, or in addition to, visually highlighting the scores (e.g., with color coding or shading). In some embodiments, when a user moves a mouse cursor over or clicks on each day/shift 302 and/or month 304, the sub-scores making up the workload burnout risk score, emotional burnout risk score, and physical burnout risk score may be shown. For example, by hovering over, clicking on, or otherwise selecting workload burnout risk score icon 306 for a particular day/shift 302, GUI 300 may display values for the underlying patient workload factor, patient medical complexity workload factor, patient demand workload factor, patient admission/discharge workload factor, medical procedure length workload factor, caregiver urgency workload factor, staffing workload factor, and/or shift length/overtime workload factor that together make up the workload burnout risk score for that day/shift 302 . GUI 300 may similarly show the values for the underlying factors when the user hovers over, clicks on, or otherwise selects emotional burnout risk score icon 308 or physical burnout risk score icon 310 for a particular day/shift 302 or month 304.

GUI 300 may provide other types of displays, as well. For example, in one embodiment, GUI 300 may show trends over time of the caregivers' total burnout risk score; workload burnout risk score, emotional burnout risk score, and/or physical burnout risk score making up the total actual burnout score; and/or the scores for factors underlying the individual sub-scores. In other examples, GUI 300 may show a list of a supervisor's clinical staff with their corresponding scores. Additionally, rather than or in addition to a calendar, GUI 300 may provide a line chart/graph displaying the scores and/or sub-scores over time, for example.

GUI 300 may also show the scores as values relative to each caregiver's baseline scores, including caregiver overall average, caregiver trailing average, and caregiver-specific burnout threshold. The caregiver-specific burnout threshold may be automatically adjusted based on automated data and/or machine learning. The threshold may also be adjusted based on manual feedback from the supervisor or caregiver. For example, feedback from questions at beginning/end of shift may asking about mood, etc. may be used to adjust the threshold.

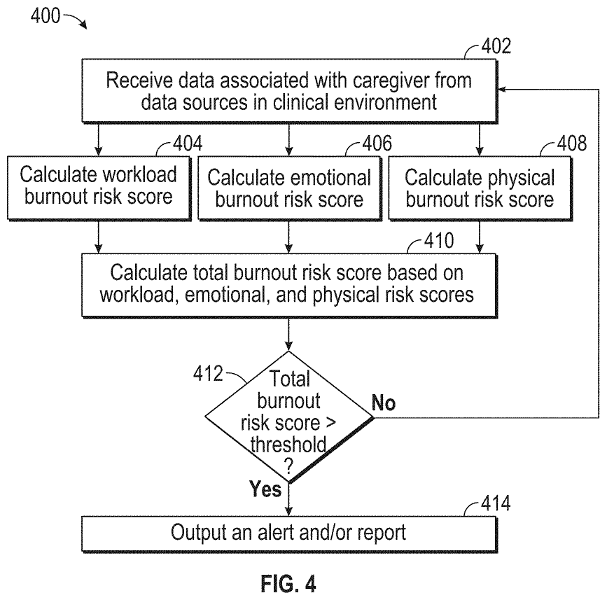

FIG. 4 illustrates an exemplary burnout risk management method 400, consistent with the disclosed embodiments. In some implementations, method 400 may be performed by processor(s) 212 of burnout risk management system 130 executing computer program instructions stored in memory 202, such as instructions associated with elements 204-210. Additionally, the steps of method 400 may be performed in orders other than that shown expressly in FIG. 4, as well as include additional and/or different steps. Method 400 may be performed using data accumulated over a shift, a number of shifts, or another period of time (e.g., a week, month, or year).

In step 402, burnout risk management system 130 may receive data associated with caregiver 102 from the clinical data sources in clinical environment 100 over network(s) 132. For example, burnout risk management system 130 may receive video data from camera(s) 108; audio data from microphone(s) 110; EHR records from EHR system 112; patient call records from caregiver call system 114; caregiver 102, patient 104, and/or visitor location information, status information, and/or identification information from caregiver communication system 116, tracking system 118, and/or wearable device 122; patient-caregiver and caregiver-family message records from caregiver communication system 116; bed exit records from bed 120 and/or load sensor 136; physiological and/or movement parameter information from wearable device 122; and/or patient lift records from patient lift system(s) 128. The data may be received for a shift, over a number of shifts, and/or over another period of time (e.g., a month or a year).

In step 404, burnout risk management system 130 may calculate a workload burnout risk score for the caregiver based on the data received in step 402. For example, based on the received data, burnout risk management system 130 may determine values for the patient workload factor, patient medical complexity workload factor, patient demand workload factor, patient admission/discharge workload factor, medical procedure length workload factor, caregiver urgency workload factor, staffing workload factor, and/or shift length/overtime workload factor, as discussed above. And burnout risk management system 130 may calculate the workload burnout risk score based on the individual values for those factors, as discussed above.

In step 406, burnout risk management system 130 may calculate an emotional burnout risk score for the caregiver based on the data received in step 402. For example, based on the received data, burnout risk management system 130 may determine values for the violent events factor, negative interaction factor, patient death factor, as discussed above. And burnout risk management system 130 may calculate the emotional burnout risk score based on the individual values for those factors, as discussed above.

In step 408, burnout risk management system 130 may calculate a physical burnout risk score for the caregiver based on the data received in step 402. For example, based on the received data, burnout risk management system 130 may determine values for the distance walked factor, time standing factor, and/or patient handling activity factor, as discussed above. And burnout risk management system 130 may calculate the physical burnout risk score based on the individual values for those factors, as discussed above.

In step 410, burnout risk management system 130 may calculate a total burnout risk score based on the workload burnout risk score calculated in step 404, the emotional burnout risk score calculated in step 406, and/or the physical burnout risk score calculated in step 408, as discussed above.

In step 412, burnout risk management system 130 may determine whether the total burnout risk score calculated in step 410 is greater than a threshold. If it is determined in step 412 that the total burnout risk score is greater than a threshold, burnout risk management system 130 may output an alert or report in step 414. For example, as discussed above, the report message may be sent by email, test message, or other means to administrator 106 and may include, for example, the total burnout risk score, the workload burnout risk score, the emotional burnout risk score, and/or the physical burnout risk score for the caregiver. In this manner, administrator 106 may take preventative measures to reduce the caregiver's risk of burnout, such as changing the caregiver's work schedule to include more time off.

Method 400 may return to step 402 if it is determined in step 418 and/or 420 that the total burnout risk score is less than a threshold.

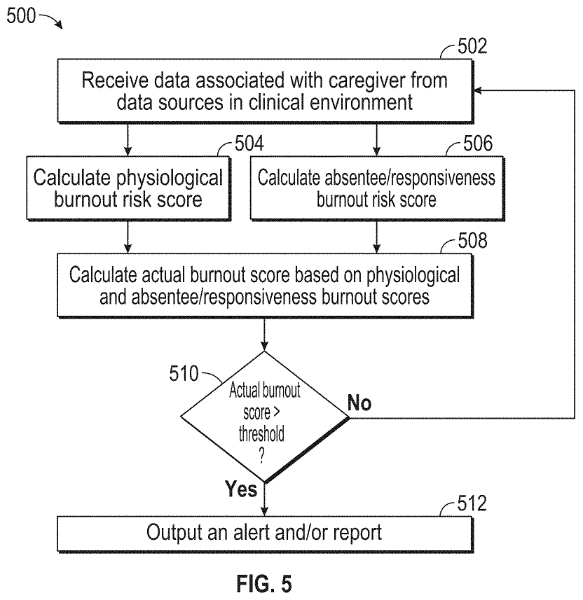

FIG. 5 illustrates an exemplary burnout management method 400, consistent with the disclosed embodiments. In some implementations, method 500 may be performed by processor(s) 212 of burnout risk management system 130 executing computer program instructions stored in memory 202, such as instructions associated with elements 204-210. Additionally, the steps of method 500 may be performed in orders other than that shown expressly in FIG. 5, as well as include additional and/or different steps.

In step 502, burnout risk management system 130 may receive data associated with caregiver 102 from the clinical data sources in clinical environment 100 over network(s) 132. For example, burnout risk management system 130 may receive video data from camera(s) 108; audio data from microphone(s) 110; EHR records from EHR system 112; patient call records from caregiver call system 114; caregiver 102, patient 104, and/or visitor location information, status information, and/or identification information from caregiver communication system 116, tracking system 118, and/or wearable device 122; patient-caregiver and caregiver-family message records from caregiver communication system 116; bed exit records from bed 120 and/or load sensor 136; physiological and/or movement parameter information from wearable device 122; and/or patient lift records from patient lift system(s) 128. The data may be received for a shift, over a number of shifts, and/or over another period of time (e.g., a month or a year).

In step 504, burnout risk management system 130 may calculate a physiological burnout score for the caregiver based on the data received in step 502. For example, burnout risk management system 130 may determine the physiological burnout score based on the heart rate data and/or skin conductivity data received from wearable device 122, as discussed above.

In step 506, burnout risk management system 130 may calculate an absentee/responsiveness burnout score for the caregiver based on the data received in step 502. For example, burnout risk management system 130 may determine the number of instances in which the caregiver is absent/tardy from the received data, and determine the absentee/responsiveness burnout score based on the number of instances.

In step 508, burnout risk management system 130 may calculate an actual burnout score based on the physiological burnout score calculated in step 504 and the absentee/responsiveness burnout score calculated in step 506, as explained above.

In step 510, burnout risk management system 130 may determine whether the actual burnout score calculated in step 508 is greater than a threshold. If it is determined in step 510 that the total burnout risk score is greater than a threshold, burnout risk management system 130 may output an alert or report in step 512. For example, as discussed above, burnout risk management system 130 may send a report message by email, text message, or other means to administrator 106 indicating that the caregiver is exhibiting signs of burnout. The report message may include, for example, the actual burnout score, the physiological burnout score, and/or the absentee /responsiveness burnout score for the caregiver. In this manner, administrator 106 may take measures to mitigate the effects of burnout on the caregiver and/or the impact on the clinical facility more broadly.

Method 500 may return to step 502 if it is determined in step 510 and/or 420 that the actual burnout risk score is less than a threshold.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

1. A method for managing caregiver burnout risk, the method comprising:
receiving, over one or more electronic communication networks from a plurality of clinical data sources in a clinical environment, data associated with a caregiver having a plurality of patients;
calculating, by one or more processors based on the data associated with the caregiver, at least one of a workload burnout risk score, an emotional burnout risk score, and physical burnout risk score for the caregiver;
calculating, by the one or more processors based on at least one of the workload burnout risk score, the emotional burnout risk score, and the physical burnout risk score for the caregiver, a burnout risk score for the caregiver;
determining, by the one or more processers, that the burnout risk score is greater than a threshold; and
generating a report based on a determination that the burnout risk score is greater than the threshold.

2. The method of claim 1, wherein the plurality of clinical data sources include one or more of a camera, a microphone, an electronic healthcare record system, a caregiver communication system, a clinical personnel/asset tracking system, a load sensor for a patient bed, a wearable device worn by the caregiver, and/or a patient lift system.

3. The method of claim 1, wherein the workload burnout risk score is calculated based on one or more of a patient workload factor indicative of a number of patients under the caregiver's care, a patient medical complexity workload factor indicative of a medical complexity of the patients under the caregiver's care, a patient demand workload factor indicative of a number of calls/requests made by the patients under the caregiver's care, patient admission/discharge workload factor indicative of a number of patients under the caregiver's care who are being admitted or discharged, a medical procedure length workload factor indicative of a length of time the caregiver is involved in a medical procure, a caregiver urgency workload factor indicative of an amount of time the caregiver spends running or rushing, a staffing workload factor indicative of a number and experience level of other clinical staff supporting the caregiver, and a shift length/overtime workload factor indicative of an amount of time worked by the caregiver.

4. The method of claim 1, wherein the emotional burnout risk score is calculated based on one or more of a violent events factor indicative of a number of violent events involving the caregiver and patients under the caregiver's care, negative interaction factor indicative of a number of negative interactions between the caregiver and family of the patients under the caregiver's care, and a patient death factor indicative of a number of deaths of the patients under the caregiver's care.

5. The method of claim 1, wherein the physical burnout risk score is calculated based on one or more of a distance walked factor indicative of a distance walked by the caregiver, a time standing factor indicative of an amount of time spend standing by the caregiver, and a patient handling activity factor indicative of a number of times the caregiver moves, lifts, or otherwise manipulates patients under the caregiver's care.

6. The method of claim 1, further comprising sending, over the one or more electronic communication networks, the report to an administrator of the clinical facility responsible for the caregiver.

7. The method of claim 1, further comprising:
causing display of a graphical user interface (GUI) including a calendar identifying days/shifts worked by the caregiver; and
indicating, for the days/shifts worked by the caregiver, the calculated at least one of the workload burnout risk score, emotional burnout risk score, and physical burnout risk score for the caregiver for the days/shifts.

8. The method of claim 7, wherein the calendar further identifies months worked by the caregiver, the method further including indicating a cumulative value for the calculated at least one of the workload burnout risk score, emotional burnout risk score, and physical burnout risk score for the caregiver for the months.

9. The method of claim 1, further comprising:
calculating, by one or more processors based on the data associated with the caregiver, at least one of a physiological burnout score and an absentee/responsiveness burnout score for the caregiver;
calculating, by the one or more processors based on at least one of the physiological burnout score and the absentee/responsiveness burnout score, an actual burnout score for the caregiver;
determining, by the one or more processers, that the actual burnout score is greater than a second threshold; and
generating a report based on a determination that the actual burnout score is greater than the second threshold.

10. A system for managing caregiver burnout risk, the system comprising:
a transceiver configured to receive, over one or more electronic communication networks from a plurality of clinical data sources in a clinical environment, data associated with a caregiver having a plurality of patients; and
one or more processors configured to:
calculate, based on the data associated with the caregiver, at least one of a workload burnout risk score, an emotional burnout risk score, and physical burnout risk score for the caregiver;
calculate, based on at least one of the workload burnout risk score, the emotional burnout risk score, and the physical burnout risk score for the caregiver, a burnout risk score for the caregiver;
determine that the burnout risk score is greater than a threshold; and
generate a report based on a determination that the burnout risk score is greater than the threshold.

11. The system of claim 10, wherein the plurality of clinical data sources include one or more of a camera, a microphone, an electronic healthcare record system, a caregiver communication system, a clinical personnel/asset tracking system, a load sensor for a patient bed, a wearable device worn by the caregiver, and/or a patient lift system.

12. The system of claim 10, wherein the one or more processors are configured to calculate the workload burnout risk score based on one or more of a patient workload factor indicative of a number of patients under the caregiver's care, a patient medical complexity workload factor indicative of a medical complexity of the patients under the caregiver's care, a patient demand workload factor indicative of a number of calls/requests made by the patients under the caregiver's care, patient admission/discharge workload factor indicative of a number of patients under the caregiver's care who are being admitted or discharged, a medical procedure length workload factor indicative of a length of time the caregiver is involved in a medical procure, a caregiver urgency workload factor indicative of an amount of time the caregiver spends running or rushing, a staffing workload factor indicative of a number and experience level of other clinical staff supporting the caregiver, and a shift length/overtime workload factor indicative of an amount of time worked by the caregiver.

13. The system of claim 10, wherein the one or more processors are configured to calculate the emotional burnout risk score based on one or more of a violent events factor indicative of a number of violent events involving the caregiver and patients under the caregiver's care, negative interaction factor indicative of a number of negative interactions between the caregiver and family of the patients under the caregiver's care, and a patient death factor indicative of a number of deaths of the patients under the caregiver's care.

14. The system of claim 10, wherein the one or more processors are configured to calculate the physical burnout risk score based on one or more of a distance walked factor indicative of a distance walked by the caregiver, a time standing factor indicative of an amount of time spend standing by the caregiver, and a patient handling activity factor indicative of a number of times the caregiver moves, lifts, or otherwise manipulates patients under the caregiver's care.

15. The system of claim 10, wherein the one or more processors are further configured to:
calculate, based on the data associated with the caregiver, at least one of a physiological burnout score and an absentee/responsiveness burnout score for the caregiver;
calculate, based on at least one of the physiological burnout score and the absentee/responsiveness burnout score, an actual burnout score for the caregiver;
determine that the actual burnout score is greater than a second threshold; and
generate a report based on a determination that the actual burnout score is greater than the second threshold.

16. A computer-readable storage medium storing instructions that, when executed by one or more processors, cause the one or more processors to perform a method for managing caregiver burnout risk, the method comprising:
receiving, over one or more electronic communication networks from a plurality of clinical data sources in a clinical environment, data associated with a caregiver having a plurality of patients;
calculating, based on the data associated with the caregiver, at least one of a workload burnout risk score, an emotional burnout risk score, and physical burnout risk score for the caregiver;
calculating, based on at least one of the workload burnout risk score, the emotional burnout risk score, and the physical burnout risk score for the caregiver, a burnout risk score for the caregiver;
determining that the burnout risk score is greater than a threshold; and
generating a report based on a determination that the burnout risk score is greater than the threshold.

17. The computer-readable storage medium of claim 16, wherein the plurality of clinical data sources include one or more of a camera, a microphone, an electronic healthcare record system, a caregiver communication system, a clinical personnel/asset tracking system, a load sensor for a patient bed, a wearable device worn by the caregiver, and/or a patient lift system.

18. The computer-readable storage medium of claim 16, wherein the workload burnout risk score is calculated based on one or more of a patient workload factor indicative of a number of patients under the caregiver's care, a patient medical complexity workload factor indicative of a medical complexity of the patients under the caregiver's care, a patient demand workload factor indicative of a number of calls/requests made by the patients under the caregiver's care, patient admission/discharge workload factor indicative of a number of patients under the caregiver's care who are being admitted or discharged, a medical procedure length workload factor indicative of a length of time the caregiver is involved in a medical procure, a caregiver urgency workload factor indicative of an amount of time the caregiver spends running or rushing, a staffing workload factor indicative of a number and experience level of other clinical staff supporting the caregiver, and a shift length/overtime workload factor indicative of an amount of time worked by the caregiver.

19. The computer-readable storage medium of claim 16, wherein the emotional burnout risk score is calculated based on one or more of a violent events factor indicative of a number of violent events involving the caregiver and patients under the caregiver's care, negative interaction factor indicative of a number of negative interactions between the caregiver and family of the patients under the caregiver's care, and a patient death factor indicative of a number of deaths of the patients under the caregiver's care.

20. The computer-readable storage medium of claim 16, wherein the physical burnout risk score is calculated based on one or more of a distance walked factor indicative of a distance walked by the caregiver, a time standing factor indicative of an amount of time spend standing by the caregiver, and a patient handling activity factor indicative of a number of times the caregiver moves, lifts, or otherwise manipulates patients under the caregiver's care.
